(19) United States
(12) Patent Application Publication
(10) Pub. No.: US 2007/0208295 A1
(43) Pub. Date: Sep. 6, 2007

(54) Double chamber syringe

(76) Inventor: Mohammadali Oloodmiyazdi, Phoenix, AZ (US)

Correspondence Address: MOHAMMADALI OLOOMIYAZDI, 1708 WEST SOUTHFORK DRIVE, PHOENIX, AZ, 85045, US

(21) Appl. No.: 11/329,925

(22) Filed: Mar. 3, 2006

(51) Int. Cl. A61M 37/00; A61M 5/00

(52) U.S. Cl. 604/82; 604/187

(57) ABSTRACT

Double chamber syringe is designed with double tasks. This kind of syringe has ability to deliver amount of medication followed by delivering the certain amount of salin flush fluid or second prescribed medication. At this moment, Those tasks are performed by two separate syringes. The advantages of using double chamber syringe are reducing the risk of infection, cost and the time spending for administration of medication. The double chamber syringe is presented in three different types.

### BACKGROUND OF INVENTION

The present invention relates to a double function syringe that can deliver prescribed medication followed by flush fluid or second prescribed medication.

At this time this process has been done with two separate syringes.

The first syringe delivers the medicine to the patient and the fluid is injected by prefilled flushing syringe. The purpose of injecting the fluid following medicine is to deliver the residual medicine remained in the intravenous tube and keeping that tube clean and also reducing the blood reflux.

According to the World Health Organization, more than 16 billion injections are given every year. Those double steps increase the risk of infection because of more human contact and increase the cost and the time spending for preparing two instead of one syringes.

### SUMMARY OF THE INVENTION

An object of the present invention is to provide a syringe with double functions.

The first function is delivering the medicine and the second function is delivering the flush fluid following the injection of the medicine.

At the present time these functions are done by two separate syringes.

The subject matter of the present invention is to decrease the risk of infection due to decreasing the human contact during preparation of syringes.

It also decrease the cost because of using one instead of two syringes and it also make the job easier and faster.

This is important specially in emergency situation or during anesthesia induction.

Also the double chamber syringe can be used to deliver two different medications at the same time or subsequently.

### BRIEF DESCRIPTION OF THE DRAWING

FIG. 1A represent the longitudinal section of type A double chamber syringe.

FIG. 1B represent the longitudinal section of detachable needle with half open-half blunt fill nub.

FIG. 2 represent the longitudinal section of type B double chamber syringe.

FIG. 3 represent the longitudinal section of type C double chamber syringe.

### DETAILED DESCRIPTION OF THE INVENTION

Type A:

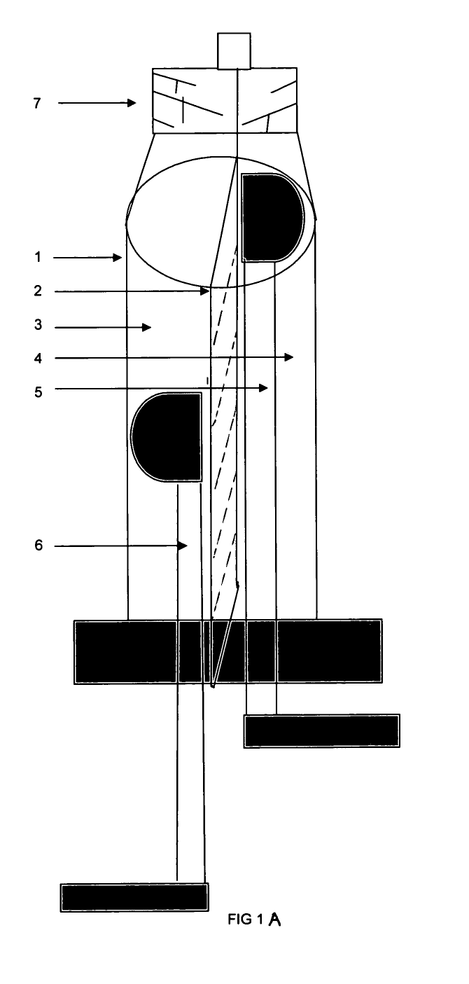

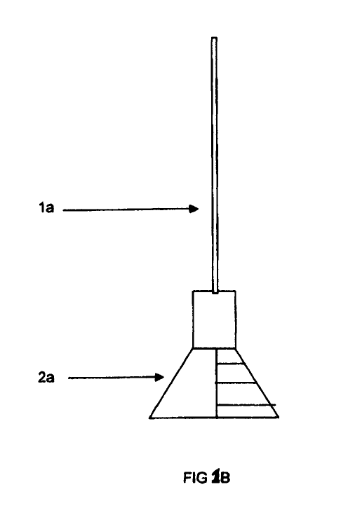

In this type of double chamber syringe, a body of syringe (1) is divided by a vertical plate (2) to two chambers. The chambers (3,4) are designed in the same or different sizes. FIG. 1A is a longitudinal view of the double chamber syringe, type A.

First chamber (3) is either prefilled by saline flush fluid or filled by medication and the second one (4) is filled by prescribed dose of medication.

A slidable plunger (5) with a rubber semi-circle shap stopper at the tip is moving into the second chamber. The prescribed medication is drawn to the second chamber by pulling back of plunger into the chamber.

At the first phase, the medication is delivered by pushing the plunger into the chamber.

At the second phase, the saline flush is delivered by pushing a plunger with a rubber semi-circle shap at the tip (6) into the first chamber (3).

In the case of using needle, a detachable needle (1a) is mounted onto luer-lok tip of syringe (7). The needle has a half open-half blunt fill nub (2a). FIG. 1B is a longitudinal sectional view of the needle.

The needle has ability to move 180 degree to two opposite directions. Therefore, first, the half open part of the nub is setting along the second chamber and medication is passing through the needle by pushing the plunger forward.

Then, the half open of the nub is setting along the first chamber by turing the needle 180 degree in the lour-lok tip and the saline flush fluid is passing through the needle by pushing the plunger forward in the first chamber.

Type B:

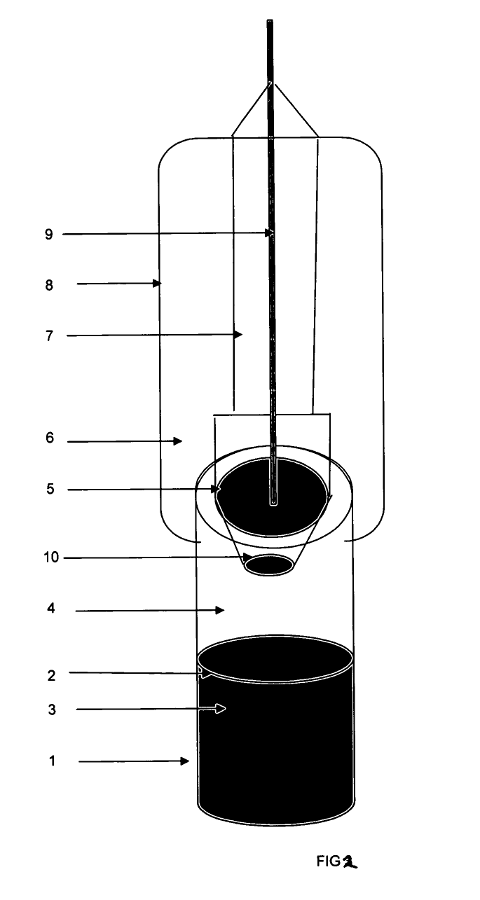

In this type of double chamber syringe, a cylider shape vial (1) is divided horizontally by a plastic membrane to two equal or unequal chambers (3,4). FIG. 2 is a longitudinal sectional view of the double chamber syring, type B.

The bottom chamber (3) is prefilled by saline flush fluid. The top chamber (4) is empty.

The vial is capped by a rubber cone shape stopper (5). The vial is threaded to an injector (6) by turnnig the stopper into the base of the injector.

The injector has a central cylinder (7) surrounded by a peripheral cylinder (8). A long needle (9) is passing through the center of injector and penetrating the stopper at the center by turning the stopper into the base of the injector.

First, the air is expelling out with pushing the vial into injector. That action is terminated before any contact between the stopper tip and the plastic membrane.

Then, the prescribed medication is drawn into the top chamber by pulling back of the vial inside the injector.

At first step, the medication is delivered by pushing the vial into the injector.

Then with more pushing the vial into the injector the plastic membrane is perforated by a sharp edge at the bottom (10) of the stopper and the saline flush fluid is delivered following administration of medication.

Type C:

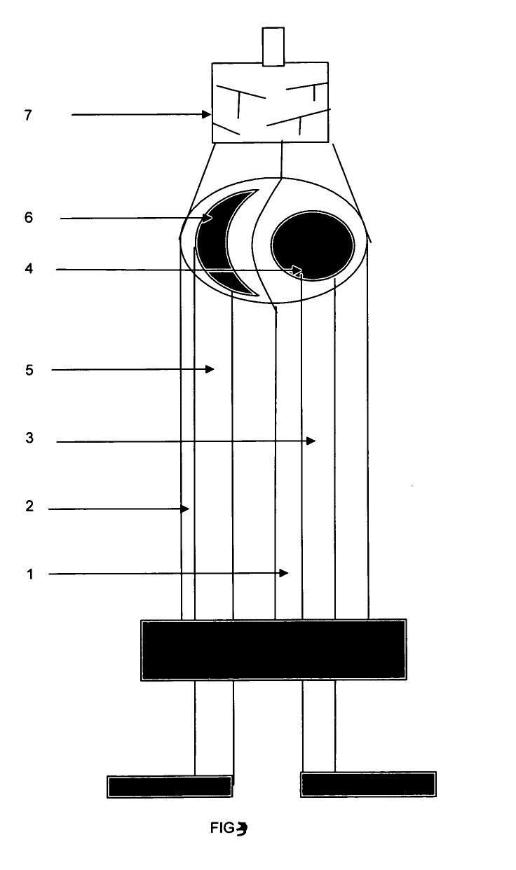

In this type of double chamber syringe having a central cylinder shape chamber (1) surrounded by a semicylinder shape chamber (2). FIG. 3A is a longitudinal sectional view of the double chamber syringe, type C.

A slidable plunger (3) with a tip covered by a rubber circle shape stopper (4) is moving inside the central chamber. The prescribed medication is drawn into the central cylinder by pulling back of the plunger.

The semicylinder chamber is prefilled with saline flush fluid. A slidable plunger (5) with a tip covered by a rubber cresent shape stopper (6) is moving into the peripheral chamber.

First, the medication is delivered br pushing the plunger forward into the central chamber.

Then, the saline flush fluid is delivered by pushing the plunger forward into the peripheral chamber following the medication administration.

The syringe having a luer-lok tip (7) that can link to a detachable needle with half open-half blunt fill nub. Although the invention has been described with respect to a preferred embodiment thereof, is not to be understood that is not to be so limited since changes and modifications can be made.

1. Type A:
A double chamber syringe type A including:
(a) a body portion divded to two semicylinder shape chambers by a vertical plate
(b) a slidable plunger with semi-circle shap tip means monuting through each said chamber
(c) a semi-circle stopper means covering the tip of each plunger
(d) a luer-lok tip mounted onto said body
(e) a detachable needle or connector having half open-half blunt fill nub link with said luer-lok tip with ability to turn 180 degree in two opposite directions means setting along said half open part of nub to one of said chambers
Type B:
A double chamber syringe type B including:
(a) a vial divided to two chambers (bottom chamber and top chamber) by a horizontal plastic membrane
(b) a stopper means for capping said vial
(c) said bottom chamber prefilled by saline or medication and said top chamber is empty
(d) an injector having a small central cylinder surrounded by a bigger peripheral cylinder
(e) a needle means extending through the center of said small central cylinder
(f) said vial threaded into injector and turned means for allowing said needle to penetrate the center of said stopper
(g) said vial pushed into said injector means for expelling out the air from said top chamber. Then, said vial pulled back from said injector means for making the vaccum and drawing medication. Finaly, said vial pushed further into said injector means for delivering said medication.
(h) a sharp edge at the bottom of said stopper means for perforating said plastic membrane by pushing said vial into said injector means expelling out salin from said bottom chamber
Type C:
A double chamber syringe type C including:
(a) a central cylinder shape chamber means for delivering prescribed medication surrounded by a semicylinder shape chamber means to deliver salin fush fluid or second prescribed medication
(b) a slidable plunger with circle shape tip means for moving back and forth into said central cylinder
(c) a slidable plunger with cresent shape tip means for moving back and forth into said semicylinder shap chamber
(d) a luer-lok tip means for linking to a needle or connector with half open-half blunt fill nub
